(19) United States
(12) Patent Application Publication
(10) Pub. No.: US 2005/0276776 A1
(43) Pub. Date: Dec. 15, 2005

(54) Cosmetic powder comprising at least one elastomeric organopolysiloxane

(76) Inventors: Anne Liechty, Paris (FR); Camille Thibout, Vincennes (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/127,279

(22) Filed: May 12, 2005

Related U.S. Application Data
(60) Provisional application No. 60/617,682, filed on Oct. 13, 2004.

(30) Foreign Application Priority Data
May 13, 2004 (FR) 04 50927

(51) Int. Cl. A61K 7/11; A61K 7/06

(52) U.S. Cl. 424/70.12; 424/70.17

(57) ABSTRACT

A cosmetic powder comprising at least one elastomeric organopolysiloxane powder, at least one spherical filler, for example, polyurethane or polymethyl methacrylate, and optionally at least one N-acylamino acid powder and use for making up and caring for the skin comprising applying the powder.

This application claims benefit of U.S. Provisional Application No. 60/617,682, filed May 13, 2004, the contents of which are incorporated herein by reference. This application also claims benefit of priority under 35 U.S.C. § 119 to French Patent Application No. 04 50927, filed May 13, 2004, the contents of which are also incorporated by reference.

Disclosed herein is a cosmetic makeup or skincare composition in powder form comprising at least one elastomeric organopolysiloxane powder combined with other powders. Further disclosed herein is a process for making up and/or caring for human skin comprising applying at least one such cosmetic composition to the skin.

In one embodiment, the cosmetic composition disclosed herein is a makeup composition. The makeup composition may be, for example, in the form of a skin makeup composition, such as a foundation, an eyeshadow, a makeup rouge, a concealer product, a face and body powder, or a body makeup product. In one embodiment, the makeup composition disclosed herein may be a foundation composition.

In another embodiment, the cosmetic composition disclosed herein is a skincare composition. The skincare composition may be in the form of a facial care product, a body care product, or a deodorant powder.

Skin makeup compositions may commonly be used to give an attractive color to the skin, such as the face, but also to mask skin imperfections, such as redness, marks and wrinkles.

Certain makeup compositions may be in the form of loose powders or compacted powders. These compositions generally comprise a high content of powders, for example, at least 80% by weight of powders, relative to the total weight of the composition. Depending on the type of powders used, the cosmetic properties of the makeup product may be very variable. However, certain powders, for example, calcium carbonate, magnesium carbonate, magnesium stearate or titanium dioxide, may give a dry, coarse feel and may be detrimental in terms of obtaining softness properties when the cosmetic powder is taken up on the finger or spread onto the skin. Furthermore, with these pulverulent materials, the deposit of the cosmetic powder on the skin may be opaque and covering, and therefore may not make it possible to obtain a transparent and masking makeup result or a makeup result that fades out the imperfections of the skin relief, for instance microreliefs, wrinkles and fine lines. The powder deposited on the skin may be very visible and may have a pronounced powdery appearance dissimilar to the natural grain of the skin: the makeup result may not be natural.

Thus, it would be desirable to provide a makeup or skincare composition in powder form that has at least one good property in terms of softness when applied to the skin and that allows a deposit, for example, a makeup result, to be obtained that is transparent and that masks the imperfections of the skin relief, for instance microreliefs, wrinkles and fine lines, while maintaining a low covering effect.

It would also be desirable to provide a cosmetic composition in powder form which allows a deposit to be obtained on the skin that is natural and not very powdery, allowing the natural grain of the skin to show through.

The present inventors have discovered that such a composition can be obtained by combining at least one elastomeric organopolysiloxane powder with other particular powders.

In one embodiment, the present disclosure relates to a cosmetic composition comprising at least one elastomeric organopolysiloxane powder, which may be cross-linked; at least one spherical filler; and at least one N-acylated amino acid powder, wherein the composition is in powder form.

In another embodiment, disclosed herein is a cosmetic composition comprising at least one elastomeric organopolysiloxane powder, which may be cross-linked, and at least one spherical filler, wherein the composition is free of compounds-chosen from calcium carbonate, magnesium carbonate, magnesium hydrocarbonate, chalks, titanium dioxide, and metal soaps derived from organic carboxylic acids comprising from 8 to 22 carbon atoms, such as 12 to 18 carbon atoms, for example, zinc stearate, magnesium stearate, lithium stearate, zinc laurate, and magnesium myristate, and wherein the composition is in powder form.

In a further embodiment, disclosed herein is a cosmetic composition comprising at least two elastomeric organopolysiloxane powders and at least one N-acylated amino acid powder, wherein the composition is in powder form.

In yet another embodiment, disclosed herein is a cosmetic composition comprising at least one elastomeric organopolysiloxane powder; at least one polymethyl methacrylate powder; and at least one N-acylated amino acid powder, wherein the composition is in powder form.

Additionally disclosed herein is a cosmetic composition comprising at least one elastomeric organopolysiloxane powder and at least one polyurethane powder, wherein the composition is in powder form.

A further embodiment disclosed herein is a cosmetic composition comprising at least one elastomeric organopolysiloxane powder and at least one acrylic polymer powder other than a polymethyl methacrylate powder, wherein the composition is in powder form.

Disclosed herein is a non-therapeutic cosmetic process for making up and/or treating the skin, comprising applying to the skin a composition disclosed herein.

Further disclosed herein is the use of a composition as disclosed herein for obtaining a deposit, for example, a makeup result, on the skin that is transparent and/or that masks the imperfections of the skin relief and/or that is natural and/or that allows the grain of the skin to show through.

The composition disclosed herein comprises at least one elastomeric organopolysiloxane powder, which may, for example, be spherical.

The at least one elastomeric organopolysiloxane may, for example, be crosslinked and may be obtained via a crosslinking addition reaction of diorganopolysiloxane comprising at least one hydrogen linked to silicon and of diorganopolysiloxane comprising ethylenically unsaturated groups linked to silicon, in the presence, for example, of a platinum catalyst; or via a dehydrogenation crosslinking condensation reaction between a diorganopolysiloxane comprising hydroxyl end groups and a diorganopolysiloxane comprising at least one hydrogen linked to silicon, in the presence of, for example, an organotin; or via a crosslinking condensation reaction of a diorganopolysiloxane comprising hydroxyl end groups and of a hydrolysable organopolysilane; or via thermal crosslinking of organopolysiloxane, for example, in the presence of an organoperoxide catalyst; or via crosslinking of organopolysiloxane by high-energy radiation such as gamma rays, ultraviolet rays or an electron beam.

In one embodiment, the at least one elastomeric organopolysiloxane powder is crosslinked and is obtained via a crosslinking addition reaction (A2) of diorganopolysiloxane comprising at least two hydrogens, each linked to a silicon, and (B2) of diorganopolysiloxane comprising at least two ethylenically unsaturated groups linked to silicon, for example, in the presence (C2) of a platinum catalyst, for instance as described in Patent Application No. EP-A-295 886.

For example, the organopolysiloxane may be obtained via a reaction of dimethylpolysiloxane comprising dimethylvinylsiloxy end groups and of methylhydrogenopolysiloxane comprising trimethylsiloxy end groups, in the presence of a platinum catalyst.

Compound (A2) is the base reagent for the formation of elastomeric organopolysiloxane and the crosslinking takes place via an addition reaction of compound (A2) with compound (B2) in the presence of the catalyst (C2).

Compound (A2) may, for example, be a diorganopolysiloxane comprising at least two lower alkenyl groups (for example C2-C4); the lower alkenyl group may be chosen from vinyl, allyl and propenyl groups. These lower alkenyl groups may be located in any position of the organopolysiloxane molecule, but in one embodiment are located at the ends of the organopolysiloxane molecule. The organopolysiloxane (A2) may have a branched-chain, linear-chain, cyclic or network structure; in one embodiment, the linear-chain structure may be used. Compound (A2) may have a viscosity ranging from the liquid state to the gum state. For example, compound (A2) may have a viscosity of at least 100 centistokes at 25° C.

The organopolysiloxanes (A2) may be chosen from methylvinylsiloxanes, methylvinylsiloxane-dimethylsiloxane copolymers, dimethylpolysiloxanes comprising dimethylvinylsiloxy end groups, dimethylsiloxane-methylphenylsiloxane copolymers comprising dimethylvinylsiloxy end groups, dimethylsiloxane-diphenylsiloxane-methylvinylsiloxane copolymers comprising dimethylvinylsiloxy end groups, dimethyl-siloxane-methylvinylsiloxane copolymers comprising trimethylsiloxy end groups, dimethylsiloxane-methylphenylsiloxane-methylvinylsiloxane copolymers comprising trimethylsiloxy end groups, methyl(3,3,3-trifluoropropyl)polysiloxane comprising dimethylvinylsiloxy end groups, and dimethylsiloxane-methyl(3,3,3-trifluoropropyl)siloxane copolymers comprising dimethylvinylsiloxy end groups.

Compound (B2) may, for example, be an organopolysiloxane comprising at least two hydrogens linked to silicon in each molecule and is thus the crosslinking agent for the compound (A2).

In one embodiment, the sum of the number of ethylenic groups per molecule of compound (A2) and the number of hydrogen atoms linked to silicon per molecule of compound (B2) is at least 4.

Compound (B2) may be of any molecular structure. In one embodiment, compounds (B2) is of linear-chain or branched-chain structure or cyclic structure.

Compound (B2) may have a viscosity at 25° C. ranging from 1 to 50 000 centistokes, for example, in order to have good miscibility with compound (A).

In one embodiment, compound (B2) may be added in an amount such that the molecular ratio between the total amount of hydrogen atoms linked to silicon in compound (B2) and the total amount of all the ethylenically unsaturated groups in compound (A2) is within the range from 1:1 to 20:1.

Compound (B2) may be chosen from methylhydrogenopolysiloxanes comprising trimethylsiloxy end groups, dimethylsiloxane-methylhydrogenosiloxane copolymers comprising trimethylsiloxy end groups, and cyclic dimethylsiloxane-methylhydrogenosiloxane copolymers.

Compound (C2) is the crosslinking reaction catalyst, and may, for example, be chosen from chloroplatinic acid, chloroplatinic acid-olefin complexes, chloroplatinic acid-alkenylsiloxane complexes, chloroplatinic acid-diketone complexes, platinum black, and platinum on a support.

The catalyst (C2) may, for example, be added in an amount ranging from 0.1 to 1000 parts by weight and, further, for example, from 1 to 100 parts by weight, as clean platinum metal, per 1000 parts by weight of the total amount of compounds (A2) and (B2).

Other organic groups may be linked to silicon in the organopolysiloxanes (A2) and (B2) described previously, for example, alkyl groups, such as methyl, ethyl, propyl, butyl or octyl; substituted alkyl groups, such as 2-phenylethyl, 2-phenylpropyl or 3,3,3-tri-fluoropropyl; aryl groups, such as phenyl, tolyl or xylyl; substituted aryl groups, such as phenylethyl; and substituted monovalent hydrocarbon-based groups, such as an epoxy group, a carboxylate ester group or a mercapto group.

In some embodiments, the at least one elastomeric organopolysiloxane powder may, for example, be chosen from non-emulsifying elastomers. As used herein, the term “non-emulsifying” means organopolysiloxane elastomers not comprising a hydrophilic chain, such as polyoxyalkylene or polyglycerolated units.

Spherical elastomeric organopolysiloxanes are, for example, described in Patent Application Nos. JP-A-61-194 009, EP-A-242 219, EP-A-295 886 and EP-A-765 656, the contents of which are incorporated by reference.

Elastomer organopolysiloxane powders that may be used include those sold under the names “Dow Corning 9505 Powder” and “Dow Corning 9506 Powder” by the company Dow Corning; these powders have the INCI name: dimethicone/vinyl dimethicone crosspolymer.

The at least one elastomeric organopolysiloxane powder may, for example, be chosen from elastomeric organopolysiloxane powders coated with silicone resin, for example, with silsesquioxane resin, as described, for example, in U.S. Pat. No. 5,538,793, the content of which is incorporated by way of reference. Such elastomeric powders are sold under the names “KSP-100”, “KSP-101”, “KSP-102”, “KSP-103”, “KSP-104” and “KSP-105” by the company Shin-Etsu, and have the INCI name: vinyl dimethicone/methicone silsesquioxane crosspolymer.

Other elastomeric organopolysiloxanes in the form of spherical powders may be powders of hybrid silicone functionalized with fluoroalkyl groups, sold, for example, under the name “KSP-200” by the company Shin-Etsu and powders of hybrid silicones functionalized with phenyl groups, sold, for example, under the name “KSP-300” by the company Shin-Etsu.

In one embodiment, the composition may, for example, comprise at least two powders of elastomeric organopolysiloxane chosen from elastomeric organopolysiloxane powders coated with silicone resin, for example, with silsesquioxane resin, as described previously.

The at least one elastomeric organopolysiloxane powder, for example, at least one non-emulsifying elastomeric organopolysiloxane, which in some embodiments may be spherical, may be present in the composition in an amount ranging from 5% to 25% by weight, for example, ranging from 7% to 15% by weight and, further, for example, ranging from 8% to 12% by weight, relative to the total weight of the composition.

In some embodiments, the composition disclosed herein may comprise at least one elastomeric organopolysiloxane powder chosen from elastomeric organopolysiloxane powders coated with silicone resin, for example, with silsesquioxane resin, in an amount ranging from 1% to 25% by weight, for example, from 1% to 15% by weight, further, for example, ranging from 2% to 8% by weight and, even further, for example, ranging from 3% to 7% by weight, relative to the total weight of the composition.

The composition disclosed herein may comprise a mixture of at least one elastomeric organopolysiloxane powder chosen from elastomeric organopolysiloxane powders coated with silicone resin, for example, with silsesquioxane resin, and uncoated elastomeric organopolysiloxane powders. In such a mixture, the elastomeric organopolysiloxane powders coated with silicone resin, for example, with silsesquioxane resin, may be present in an amount ranging from 1% to 10% by weight, for example, ranging from 2% to 8% by weight and, further, for example, ranging from 3% to 7% by weight, relative to the total weight of the composition; the uncoated elastomeric organopolysiloxane powders may be present in an amount ranging from 1% to 10% by weight, for example, ranging from 2% to 8% by weight and, further, for example, ranging from 3% to 7% by weight, relative to the total weight of the composition.

The composition disclosed herein may comprise at least one spherical filler. The at least one spherical filler may be chosen from mineral and organic fillers, and in one embodiment the at least one spherical filler is chosen from organic fillers. The at least one spherical filler is different from the at least one elastomeric organopolysiloxane powder described previously. The at least one spherical filler may, for example, be non-elastomeric. In some embodiments, the at least one spherical filler is not film-forming, i.e. it does not form a continuous film when deposited onto a support such as the skin.

The at least one spherical filler may be chosen, for example, from: acrylic powders, for example, polymethyl methacrylate powders, acrylic powders other than polymethyl methacrylate powders, polyurethane powders, and the powders described below.

The composition disclosed herein may comprise the at least one spherical filler in an amount ranging from 10% to 40% by weight, for example, ranging from 15% to 35% by weight, further, for example, ranging from 15% to 30% by weight and, even further, for example, ranging from 15% to 25% by weight, relative to the total weight of the composition.

According to one embodiment, the composition may comprise at least one powder, for example, a spherical powder, of polymethyl methacrylate.

The at least one polymethyl methacrylate powder may be in the form of hollow or solid white spherical particles generally with a number-average size of micrometer order, for example, ranging from 3 to 15 microns and, further, for example, ranging from 3 to 10 microns. As used herein, the expression “number-average size” means the size given by the statistical particle size distribution to half of the population, referred to as D50.

It is also possible to characterize the polymethyl methacrylate particles by their density, which can vary, for example, as a function of the size of the spherical cavity of the particles.

In accordance with the embodiments disclosed herein, this density is assessed according to the following protocol, referred to as the packed density:
m=40 g of powder is poured into a measuring cylinder; the measuring cylinder is then placed on a Stav 2003 machine from Stampf Volumeter; the measuring cylinder is then subjected to 1500 packing motions; the final volume Vf of packed powder is then measured directly on the measuring cylinder. The packed density is determined by the ratio m/Vf, in this instance 40/Vf (Vf being expressed in cm³ and m in g).

For example, the density of the at least one polymethyl methacrylate powder that may be used in the embodiments disclosed herein may range, for example, from 0.3 to 1.5, further, for example, from 0.5 to 1.5 and, even further, for example, from 1 to 1.5.

As non-limiting illustrations of the at least one polymethyl methacrylate powder that is suitable for use in the composition disclosed herein, mention may be made, for example, of the polymethyl methacrylate particles sold by the company Matsumoto Yushi Co. under the name “Micropearl M100”, by the company LCW under the name “Covabead LH 85” and those sold by the company Nihon Junyaku under the name “Jurymer MB1”.

The at least one polymethyl methacrylate powder may be present in an amount ranging from 5% to 20% by weight, for example, ranging from 7% to 18% by weight and further, for example, ranging from 8% to 15% by weight, relative to the total weight of the composition.

The composition disclosed herein may comprise at least one powder, for example, a spherical powder, of an acrylic polymer, other than a polymethyl methacrylate powder.

For example, the at least one acrylic powder may be chosen from acrylonitrile polymer and acrylonitrile copolymer powders, and, for example, expanded hollow particles of acrylonitrile polymer or copolymer. For example, the particles may be made of any expanded acrylonitrile polymer or copolymer that is non-toxic and a non-irritant to the skin.

For example, the mass per unit volume of the particles is chosen in the range from 15 kg/m³ to 200 kg/m³, for example, from 40 kg/m³ to 120 kg/m³ and even further, for example, from 60 kg/m³ to 80 kg/m³. To obtain this low mass per unit volume, expanded polymer or copolymer particles, for example, based on acrylonitrile and on an acrylic or styrene monomer and/or on vinylidene chloride may, for example, be used.

It is possible to use, for example, a copolymer comprising: from 0% to 60% of units derived from vinylidene chloride, from 20% to 90% of units derived from acrylonitrile and from 0% to 50% of units derived from an acrylic or styrene monomer, wherein the sum of the percentages (by weight) is equal to 100. The acrylic monomer may, for example, be a methyl or ethyl acrylate or methacrylate. The styrene monomer may, for example, be α-methylstyrene or styrene.

In one embodiment, the particles used in the composition disclosed herein are chosen from hollow particles of an expanded copolymer of vinylidene chloride and of acrylonitrile or of vinylidene chloride and of acrylonitrile and of methacrylate. These particles may be dry or hydrated.

The particles may be obtained, for example, according to the processes disclosed in Patent and Patent Application Nos. EP 56 219, EP 348 372, EP 486 080, EP 320 473, EP 112 807 and U.S. Pat. No. 3,615,972.

The internal cavity of the particles in principle comprises at least one gas, which may be chosen from air, nitrogen, and hydrocarbons, such as isobutane and isopentane.

In some embodiments, the particles disclosed herein have a particle size ranging from 1 μm to 80 μm, for example, ranging from 10 μm to 50 μm and from 10 μm to 30 μm.

The particles may be chosen, for example, from expanded terpolymer micro-spheres of vinylidene chloride, of acrylonitrile and of methacrylate, sold under the brand name Expancel by the company Expancel under the references 551 DE 50 (particle size of 40 μm), 551 DE 20 (particle size of 30 μm and mass per unit volume of 65 kg/m³), 551 DE 12 (particle size of 12 μm), 551 DE 80 (particle size of 80 μm) and 461 DE 50 (particle size of 50 μm). It is also possible to use microspheres formed from the same expanded terpolymer having a particle size of 8 μm and a mass per unit volume of 70 kg/m³, referred to hereinbelow as EL 23, or having a particle size of 34 μm and a mass per unit volume of 20 kg/m³, referred to hereinbelow as EL 43.

In some embodiments, the at least one acrylic powder is present in the composition disclosed herein when it is in the form of a loose powder.

The at least one acrylic powder other than polymethyl methacrylate powder may be present in the composition disclosed herein in an amount ranging from 0.05% to 2% by weight, for example, ranging from 0.1% to 1.5% by weight, and, further, for example, ranging from 0.1% to 1.2% by weight, relative to the total weight of the composition.

The composition disclosed herein may comprise at least one powder, for example, a spherical powder, of polyurethane. In one embodiment, the at least one polyurethane powder is not film-forming, i.e. it does not form a continuous film when it is deposited onto a support such as the skin.

For example, the at least one polyurethane powder is a powder of a copolymer of hexamethylene diisocyanate and trimethylol hexyl lactone. Such a polyurethane powder is sold, for example, under the names “Plastic Powder D-400” and “Plastic Powder D-800” by the company Toshiki.

Other polyurethane powders that may be used include the product sold under the name “Plastic Powder CS-400” by the company Toshiki.

The at least one polyurethane powder may be present in the composition disclosed herein in an amount ranging from 0.5% to 30% by weight, for example, ranging from 1% to 15% by weight and, further, for example, ranging from 5% to 15% by weight, relative to the total weight of the composition.

The composition disclosed herein may comprise at least one N-acylated amino acid powder. Such a powder may give a cosmetic powder a creamy property.

The at least one N-acylated amino acid powder comprises at least one acyl group comprising from 8 to 22 carbon atoms, for example, a 2-ethylhexanoyl, caproyl, lauroyl, myristoyl, palmitoyl, stearoyl, or cocoyl group. The amino acid may be, for example, lysine, glutamic acid, or alanine.

The at least one N-acylated amino acid powder may, for example, be a lauroyllysine powder.

The at least one N-acylated amino acid powder may be present in the composition disclosed herein in an amount ranging from 5% to 20% by weight, for example, ranging from 7% to 18% by weight and, further, for example, ranging from 8% to 15% by weight, relative to the total weight of the composition.

The composition disclosed herein may comprise barium sulfate. The barium sulfate particles may be coated with at least one N-acylated amino acid powder, such as those described previously, for example, coated with lauroyllysine.

The barium sulfate may be present in the composition disclosed herein in an amount ranging from 1% to 10% by weight, for example, from 2% to 8% by weight, and, further, for example, from 3% to 7% by weight, relative to the total weight of the composition.

The composition disclosed herein may comprise at least one pulverulent dyestuff, which may be chosen from pigments and nacres.

As used herein, the term “pigments” should be understood as meaning white or colored, mineral or organic particles of any shape, which are insoluble in the physiological medium, and which are intended to color the composition.

As used herein, the term “nacres” should be understood as meaning iridescent particles of any shape, for example, produced in the shell of certain molluscs or alternatively synthesized.

The pigments may be white or colored, and mineral and/or organic. Among the mineral pigments that may be mentioned are titanium dioxide, optionally surface-treated, zirconium oxide or cerium oxide, and also zinc oxide, iron oxide (black, yellow or red) or chromium oxide, manganese violet, ultramarine blue, chromium hydrate and ferric blue, and metal powders, such as aluminium powder or copper powder.

Among the organic pigments that may be mentioned are carbon black, pigments of D & C type, and lakes based on cochineal carmine or on barium, strontium, calcium or aluminium.

The nacreous pigments may be chosen from white nacreous pigments such as mica coated with titanium or with bismuth oxychloride, colored nacreous pigments, such as titanium mica coated with iron oxides, titanium mica coated, for example, with ferric blue or with chromium oxide, titanium mica coated with an organic pigment of the abovementioned type, and also nacreous pigments based on bismuth oxychloride.

It is also possible to use goniochromatic pigments; these pigments exhibit a relatively large color change according to the angle of observation.

The goniochromatic pigment may be chosen, for example, from pigments of multilayer interference structure and liquid-crystal pigments.

In the case of a multilayer structure, this structure may comprise, for example, at least two layers, each layer, independently of the other layer(s) or otherwise, being made, for example, from at least one material chosen from the following materials: MgF₂, CeF₃, ZnS, ZnSe, Si, SiO₂, Ge, Te, Fe₂O₃, Pt, Va, Al₂O₃, MgO, Y₂O₃, S₂O₃, SiO, HfO₂, ZrO₂, CeO₂, Nb₂O₅, Ta₂O₅, TiO₂, Ag, Al, Au, Cu, Rb, Ti, Ta, W, Zn, MoS₂, cryolite, alloys, and polymers.

The goniochromatic agents with multilayer structures are, for example, those described in the following documents: U.S. Pat. No. 3,438,796, EP-A-227.423, U.S. Pat. No. 5,135,812, EP-A-170 439, EP-A-341 002, U.S. Pat. No. 4,930,866, U.S. Pat. No. 5,641,719, EP-A-472 371, EP-A-395 410, EP-A-753 545, EP-A-768 343, EP-A-571 836, EP-A-708 154, EP-A-579 091, U.S. Pat. No. 5,411,586, U.S. Pat. No. 5,364,467, WO-A-97/39066, DE-A-4 225 031, WO 95/17479 (BASF), and DE-A-196 14 637. They are in the form of flakes, of metallized color.

The multilayer structures that may be used are, for example, the following structures: Al/SiO₂/Al/SiO₂/Al; Cr/MgF₂/Al/MgF₂/Al; MoS₂/SiO₂/Al/SiO₂/MoS₂; Fe₂O₃/SiO₂/Al/SiO₂/Fe₂O₃; Fe₂O₃/SiO₂/Fe₂O₃/SiO₂/Fe₂O₃; MoS₂/SiO₂/mica-oxide/SiO₂/MoS₂; and Fe₂O₃/SiO₂/mica-oxide/SiO₂/Fe₂O₃. Different colors may be obtained depending on the thickness of the various layers. Thus, with the structure Fe₂O₃/SiO₂/Al/SiO₂/Fe₂O₃, the color changes from green-golden to red-grey for SiO₂ layers ranging from 320 to 350 nm; from red to golden for SiO₂ layers ranging from 380 to 400 nm; from violet to green for SiO₂ layers ranging from 410 to 420 nm; and from copper to red for SiO₂ layers ranging from 430 to 440 nm.

Consequently, the multilayer structure may be essentially mineral or organic. Different colors are obtained depending on the thickness of each of the various layers.

The goniochromatic pigments of multilayer interference structure according to the embodiments disclosed herein are, for example, those described in the following documents: U.S. Pat. No. 3,438,796, EP-A-227 423, U.S. Pat. No. 5,135,812, EP-A-170 439, EP-A-341 002, U.S. Pat. No. 4,930,866, U.S. Pat. No. 5,641,719, EP-A-472 371, EP-A-395 410, EP-A-753 545, EP-A-768 343, EP-A-571 836, EP-A-708 154, EP-A-579 091, U.S. Pat. No. 5,411,586, U.S. Pat. No. 5,364,467, WO-A-97/39066, DE-A4 225 031, WO 95/17479 (BASF), DE-A-196 14 637, and combinations thereof. They may be in the form of flakes, of metallized color.

In one embodiment, the goniochromatic pigment of multilayer interference structure may be chosen from the following commercial goniochromatic pigments: Infinite Colors from the company Shiseido, Sicopearl Fantastico from BASF, Colorstream, Xirallic or Xirona from Merck, Colorglitter from Flex, and mixtures thereof.

As goniochromatic pigments of multilayer structure, mention may be made of those sold under the name “Sicopearl”.

Liquid-crystal pigments are, for example, described in Patent Application No. EP-A-1 046 692.

Liquid-crystal particles that may, for example, be used include those known under the CTFA name Polyacrylate-4 and sold under the names “Helicone® HC Sapphire”, “Helicone® HC Scarabeus”, “Helicone® HC Jade”, “Helicone® HC Maple”, “Helicone® HC XL Sapphire”, “Helicone® HC XL Scarabeus”, “Helicone® HC XL Jade” and “Helicone® HC XL Maple” by the company Wacker.

The at least one pulverulent dyestuff may be present in the composition disclosed herein in an amount ranging from 0.5% to 40% by weight, for example, ranging from 1% to 30% by weight and, further, for example, ranging from 3% to 25% by weight, relative to the total weight of the composition.

The composition disclosed herein may comprise at least one additional filler, other than the at least one elastomeric organopolysiloxane powder and the at least one spherical filler described previously.

As used herein, the term “fillers” should be understood as meaning colorless or white, mineral or synthetic particles of any shape, which are insoluble in the medium of the composition irrespective of the temperature at which the composition is manufactured.

The at least one additional filler may be mineral or organic and of any shape, platelet-shaped, spherical or oblong, irrespective of the crystallographic form (for example lamellar, cubic, hexagonal, orthorhombic, etc.). Mention may be made of talc, mica, silica, kaolin, polyamide (Nylon®) powders, poly-β-alanine powders, polyethylene powders, tetrafluoroethylene polymer (Teflon®) powders, starch, boron nitride, silicone resin powders (for example Tospearls® from Toshiba), hydroxyapatite, sericite, glass beads and ceramic beads.

The at least one additional filler may be present in the composition disclosed herein in an amount ranging from 0.5% to 75% by weight, for example, ranging from 1% to 60% by weight, and, further, for example, ranging from 5% to 60% by weight, relative to the total weight of the composition.

In one embodiment, the composition disclosed herein is free of compounds chosen from calcium carbonate, magnesium carbonate, magnesium hydrocarbonate, chalks, titanium dioxide, and metal soaps derived from organic carboxylic acids comprising from 8 to 22 carbon atoms and, for example, from 12 to 18 carbon atoms, for example zinc stearate, magnesium stearate, lithium stearate, zinc laurate, and magnesium myristate. Such compounds have the drawback of harming the soft feel of the cosmetic powder.

The term “free of” means that the composition comprises no more than 4% by weight of the compounds from which it is free, for example, no more than 3% by weight, further, for example, no more than 2% by weight, further, for example, no more than 1% by weight, and even further, for example, does not comprise any (i.e. 0% by weight) of such compounds, relative to the total weight of the composition.

The composition disclosed herein may, for example, comprise a total content of pulverulent compounds ranging from 80% to 99% by weight and, further, for example, ranging from 85% to 99% by weight, relative to the total weight of the composition.

The composition disclosed herein may, for example, comprise at least one fatty phase, which may comprise at least one oil. This type of fatty phase is also commonly referred to as a binder, and serves, for example, as a dispersing medium for the particulate phase.

The at least one oil may be chosen from the oils conventionally used as a binder in loose or compact powders. For example, the at least one oil may be chosen from:
mink oil, turtle oil, soybean oil, grapeseed oil, sesame seed oil, corn oil, rapeseed oil, sunflower oil, cottonseed oil, avocado oil, olive oil, castor oil, jojoba oil, and groundnut oil;
hydrocarbon oils, such as liquid paraffin, squalane, and petroleum jelly;
fatty esters, such as isopropyl myristate, isopropyl palmitate, butyl stearate, isodecyl stearate, isocetyl stearate, hexyl laurate, isononyl isononanoate, 2-ethylhexyl palmitate, 2-hexyldecyl laurate, 2-octyldecyl palmitate, 2-octyldodecyl myristate, and lactate, 2-diethylhexyl succinate, diisostearyl malate, glyceryl triisostearate, and diglyceryl triisostearate;
silicone oils, such as polymethylsiloxanes, polymethylphenylsiloxanes, polysiloxanes modified with fatty acids, with fatty alcohols or with polyoxyalkylenes, fluoro silicones, and perfluoro oils;
higher fatty acids, such as myristic acid, palmitic acid, stearic acid, behenic acid, oleic acid, linoleic acid, linolenic acid, and isostearic acid;
higher fatty alcohols, such as cetanol, stearyl alcohol, and oleyl alcohol; and
poly methylfluoroalkyl dimethylsiloxanes of formula (I): 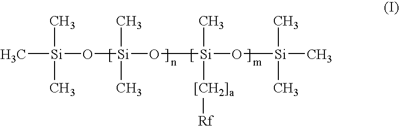
   wherein:
n is an integer ranging from 5 to 90, for example, from 30 to 80 and, further, for example, from 50 to 80;
m is an integer ranging from 1 to 150, for example, from 1 to 80 and, further, for example, from 1 to 40;
a is an integer ranging from 0 to 5, and
   Rf is chosen from perfluoroalkyl radicals comprising from 1 to 8 carbon atoms.

Examples of compounds of formula (I) include those sold under the names X22-819, X22-820, X22-821 and X22-822 by the company Shin-Etsu.

The composition disclosed herein may comprise at least one oil in an amount ranging from 1% to 20% by weight and, further, for example, from 2% to 15% by weight, relative to the total weight of the composition.

The composition may comprise at least one other common cosmetic ingredient, which may be chosen, for example, from antioxidants, fragrances, preserving agents, neutralizers, surfactants, waxes, water, sunscreens, vitamins, moisturizers, self-tanning compounds, and antiwrinkle active agents.

Needless to say, a person skilled in the art will take care to select this or these optional additional compound(s), and/or the amount thereof, such that the advantageous properties of the composition disclosed herein are not, or are not substantially, adversely affected by the envisaged addition(s).

In one embodiment, the composition disclosed herein is an anhydrous composition. As used herein, the term “anhydrous composition” means a composition comprising no more than 2% by weight of water, for example, no more than 0.5% of water, and, for example, free of water, wherein the water is not added during the preparation of the composition, but corresponding to the residual water provided by the mixed ingredients.

The composition disclosed herein may be in the form of a loose powder or a compact powder. As use herein, the term “compact powder” means a powder pressed using a manual or mechanical press.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the embodiments disclosed herein. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosed embodiments are approximations, unless otherwise indicated the numerical values set forth in the specific examples are reported as precisely as possible. Any numerical value, however, inherently contain certain errors necessarily resulting from the standard deviation found in their respective testing measurements.

The embodiments disclosed herein are illustrated in greater detail by the examples described below.

### EXAMPLE 1

A loose face powder having the following composition was prepared:

| | | |
|---|---|---|
| Particles of crosslinked polydimethylsiloxane coated | 5 | g |
| with silicone resin (KSP-100 from the company Shin-Etsu) | | |
| Spherical powder of elastomeric silicone | 5 | g |
| (Dow Corning 9506 Powder from Dow Corning) | | |
| Polyurethane spherical powder | 10 | g |
| (Plastic Powder D-400 from Toshiki) | | |
| Spherical polymethyl methacrylate powder | 10 | g |
| (Ganzpearl GMP 0820 from Ganz Chemical) | | |
| Microspheres of vinylidene chloride/acrylonitrile/methyl | 0.5 | g |
| methacrylate copolymer expanded with isobutane | | |
| (Expancel 551 DE 20 D60 from the company Expancel) | | |
| Lauroyllysine | 5 | g |
| Lauroyllysine-coated barium sulfate | 5 | g |
| (LLD-5 BaSO4 from Daito Kasei) | | |
| Talc | 37.6 | g |
| Mica | 15 | g |
| Iron oxides | 3.30 | g |
| Preserving agents | 0.6 | g |
| Isocetyl stearate | 3 | g |

The powder spread easily on the face and gave good softness properties. The makeup result obtained was transparent, homogeneous, unifying, had a natural appearance that allowed the grain of the skin to show through, and faded out the relief defects of the skin.

### EXAMPLE 2

A loose face powder having the following composition was prepared:

| | | |
|---|---|---|
| Particles of crosslinked polydimethylsiloxane coated with | 5 | g |
| silicone resin (KSP-100 from the company Shin-Etsu) | | |
| Spherical powder of elastomeric silicone | 5 | g |
| (Dow Corning 9506 Powder from Dow Corning) | | |
| Polyurethane spherical powder | 10 | g |
| (Plastic Powder D-400 from Toshiki) | | |
| Spherical polymethyl methacrylate powder | 10 | g |
| (Ganzpearl GMP 0820 from Ganz Chemical) | | |
| Microspheres of vinylidene chloride/acrylonitrile/methyl | 0.5 | g |
| methacrylate copolymer expanded with isobutane | | |
| (Expancel 551 DE 20 D60 from the company Expancel) | | |
| Lauroyllysine | 5 | g |
| Lauroyllysine-coated barium sulfate | 5 | g |
| (LLD-5 BaSO4 from Daito Kasei) | | |
| Talc | 12.7 | g |
| Mica | 12 | g |
| Iron oxides | 5.2 | g |
| Nacreous pigments | 26 | g |
| Preserving agents | 0.6 | g |
| Isocetyl stearate | 3 | g |

The powder spread easily on the face and gave good softness properties. The makeup result obtained was transparent, homogeneous, unifying, had a natural appearance that allowed the grain of the skin to show through, and faded out the relief defects of the skin.

### EXAMPLE 3

A loose face powder having the following composition was prepared:

| | | |
|---|---|---|
| Particles of crosslinked polydimethylsiloxane coated with | 20 | g |
| silicone resin (KSP-100 from the company Shin-Etsu) | | |
| Polyurethane spherical powder | 10 | g |
| (Plastic Powder D-400 from Toshiki) | | |
| Spherical polymethyl methacrylate powder | 10 | g |
| (Ganzpearl GMP 0820 from Ganz Chemical) | | |
| Microspheres of vinylidene chloride/acrylonitrile/methyl | 0.50 | g |
| methacrylate copolymer expanded with isobutane | | |
| (Expancel 551 DE 20 D60 from the company Expancel) | | |
| Lauroyllysine | 5 | g |
| Lauroyllysine-coated barium sulfate | 5 | g |
| (LLD-5 BaSO4 from Daito Kasei) | | |
| Talc | 27.6 | g |
| Mica | 15 | g |
| Iron oxides | 3.30 | g |
| Preserving agents | 0.6 | g |
| Isocetyl stearate | 3 | g |

The powder spread easily on the face and gave good softness properties. The makeup result obtained was transparent, homogeneous, unifying, had a natural appearance that allowed the grain of the skin to show through, and faded out the relief defects of the skin.

### EXAMPLE 4

A compact face powder having the following composition was prepared:

| | | |
|---|---|---|
| Particles of crosslinked polydimethylsiloxane coated with | 5 | g |
| silicone resin (KSP-100 from the company Shin-Etsu) | | |
| Elastomeric silicone spherical powder | 5 | g |
| (Dow Corning 9506 Powder from Dow Corning) | | |
| Polyurethane spherical powder | 10 | g |
| (Plastic Powder D-400 from Toshiki) | | |
| Spherical polymethyl methacrylate powder | 10 | g |
| (Ganzpearl GMP 0820 from Ganz Chemical) | | |
| Microspheres of vinylidene chloride/acrylonitrile/methyl | 0.50 | g |
| methacrylate copolymer expanded with isobutane | | |
| (Expancel 551 DE 20 D60 from the company Expancel) | | |
| Lauroyllysine | 5 | g |
| Lauroyllysine-coated barium sulfate | 5 | g |
| (LLD-5 BaSO4 from Daito Kasei) | | |
| Talc | 21.2 | g |
| Mica | 10 | g |
| Iron oxides | 3.30 | g |
| Zinc stearate | 3 | g |
| Bismuth oxychloride | 10 | g |
| Preserving agents | 1.4 | g |
| Phenyltrimethicone | 6 | g |
| Glyceryl triisostearate | 6 | g |

The powder was sieved, poured into a metal dish and then compacted.

The powder spread easily on the face and gave good softness properties. The makeup result obtained was transparent, homogeneous, unifying, had a natural appearance that allowed the grain of the skin to show through, and faded out the relief defects of the skin.

1. A cosmetic composition comprising,
at least one elastomeric organopolysiloxane powder;
at least one spherical filler; and
at least one N-acylated amino acid powder,
wherein the composition is in powder form.

2. A cosmetic composition comprising,
at least one elastomeric organopolysiloxane powder and
at least one spherical filler,
wherein the composition is free of compounds chosen from calcium carbonate, magnesium carbonate, magnesium hydrocarbonate, chalks, titanium dioxide, and metal soaps derived from organic carboxylic acids comprising from 8 to 22 carbon atoms, and
wherein the composition is in powder form.

3. A cosmetic composition comprising,
at least two elastomeric organopolysiloxane powders and
at least one N-acylated amino acid powder,
wherein the composition is in powder form.

4. A cosmetic composition comprising,
at least one elastomeric organopolysiloxane powder;
at least one polymethyl methacrylate powder; and
at least one N-acylated amino acid powder,
wherein the composition is in powder form.

5. A cosmetic composition comprising,
at least one elastomeric organopolysiloxane powder and
at least one polyurethane powder, wherein the composition is in powder form.

6. A cosmetic composition comprising,
at least one elastomeric organopolysiloxane powder and
at least one acrylic polymer powder other than a polymethyl methacrylate powder, wherein the composition is in powder form.

7. The composition according to claim 1, wherein the at least one elastomeric organopolysiloxane powder is chosen from elastomeric organopolysiloxanes obtained via at least one reaction chosen from:
a crosslinking addition reaction of diorganosiloxane comprising at least one hydrogen linked to silicon and of diorganopolysiloxane comprising ethylenically unsaturated groups linked to silicon;
a dehydrogenation crosslinking condensation reaction between a diorganopolysiloxane comprising hydroxyl end groups and a diorganopolysiloxane comprising at least one hydrogen linked to silicon;
a crosslinking condensation reaction of a diorganopolysiloxane comprising hydroxyl end groups and of a hydrolysable organopolysilane;
thermal crosslinking of organopolysiloxane; and
crosslinking of organopolysiloxane by high-energy radiation.

8. The composition according to claim 7, wherein the at least one elastomeric organopolysiloxane powder is obtained via a crosslinking addition reaction of
(A) diorganopolysiloxane comprising at least two hydrogens each linked to a silicon, and
(B) diorganopolysiloxane comprising at least two ethylenically unsaturated groups linked to silicon.

9. The composition according to claim 8, wherein the at least one elastomeric organopolysiloxane powder is obtained by reaction of dimethylpolysiloxane comprising dimethylvinylsiloxy end groups and of methylhydrogenopolysiloxane comprising trimethylsiloxy end groups, in the presence of a platinum catalyst.

10. The composition according to claim 1, wherein the at least one elastomeric organopolysiloxane powder is crosslinked and chosen from non-emulsifying elastomers.

11. The composition according to claim 1, wherein the at least one elastomeric organopolysiloxane powder is chosen from elastomeric organopolysiloxane powders coated with silicone resin.

12. The composition according to claim 1, wherein the at least one elastomeric organopolysiloxane powder is a spherical powder.

13. The composition according to claim 1, wherein the at least one elastomeric organopolysiloxane powder is present in an amount ranging from 5% to 25% by weight, relative to the total weight of the composition.

14. The composition according to claim 13, wherein the at least one elastomeric organopolysiloxane powder is present in an amount ranging from 8% to 12% by weight, relative to the total weight of the composition.

15. The composition according to claim 11, wherein the at least one elastomeric organopolysiloxane powder coated with silicone resin is present in an amount ranging from 1% to 25% by weight, relative to the total weight of the composition.

16. The composition according to claim 15, wherein the at least one elastomeric organopolysiloxane powder coated with silicone resin is present in an amount ranging from 3% to 7% by weight, relative to the total weight of the composition.

17. The composition according to claim 1, wherein the composition comprises a mixture of
at least one elastomeric organopolysiloxane powder coated with silicone resin and
at least one uncoated elastomeric organopolysiloxane powder.

18. The composition according to claim 17, wherein the at least one uncoated elastomeric organopolysiloxane powder is present in an amount ranging from 1% to 10% by weight, relative to the total weight of the composition.

19. The composition according to claim 18, wherein the at least one uncoated elastomeric organopolysiloxane powder is present in an amount ranging from 3% to 7% by weight, relative to the total weight of the composition.

20. The composition according to claim 17, wherein the at least one elastomeric organopolysiloxane powder coated with silicone resin is present in an amount ranging from 1% to 10% by weight, relative to the total weight of the composition.

21. The composition according to claim 20, wherein the at least one elastomeric organopolysiloxane powder coated with silicone resin is present in an amount ranging from 3% to 7% by weight, relative to the total weight of the composition.

22. The composition according to claim 3, further comprising at least one spherical filler.

23. The composition according to claim 1, wherein the at least one spherical filler is chosen from organic fillers.

24. The composition according to claim 1, wherein the at least one spherical filler is chosen from polymethyl methacrylate powders, acrylic powders other than polymethyl methacrylate powders, and polyurethane powders.

25. The composition according to claim 1, wherein the at least one spherical filler is present in an amount ranging from 10% to 40% by weight, relative to the total weight of the composition.

26. The composition according to claim 25, wherein the at least one spherical filler is present in an amount ranging from 15% to 25% by weight, relative to the total weight of the composition.

27. The composition according to claim 1, further comprising a polymethyl methacrylate powder.

28. The composition according to claim 27, wherein the polymethyl methacrylate powder has a density ranging from 0.3 to 1.5.

29. The composition according to claim 28, wherein the polymethyl methacrylate powder has a density ranging from 1 to 1.5.

30. The composition according to claim 1, wherein the polymethyl methacrylate powder is present in an amount ranging from 5% to 20% by weight, relative to the total weight the composition.

31. The composition according to claim 30, wherein the polymethyl methacrylate powder is present in an amount ranging from 8% to 15% by weight, relative to the total weight of the composition.

32. The composition according to claim 1, further comprising at least one acrylic polymer powder other than a polymethyl methacrylate powder.

33. The composition according to claim 6, wherein the at least one acrylic polymer powder is chosen from powders of acrylonitrile polymer and powders of acrylonitrile copolymer.

34. The composition according to claim 6, wherein the at least one acrylic polymer powder comprises expanded hollow particles of acrylonitrile polymer or of acrylonitrile copolymer.

35. The composition according to claim 6, wherein the at least one acrylic polymer powder comprises particles with a mass per unit volume ranging from 15 kg/m³ to 200 kg/m³.

36. The composition according to claim 35, wherein the at least one acrylic polymer powder comprises particles with a mass per unit volume ranging from 60 kg/m³ to 80 kg/m³.

37. The composition according to claim 6, wherein the at least one acrylic polymer powder is chosen from copolymers of acrylonitrile and of an acrylic or styrene monomer and/or of vinylidene chloride.

38. The composition according to claim 6, wherein the at least one acrylic polymer powder is a copolymer comprising: from 0% to 60% of units derived from vinylidene chloride, from 20% to 90% of units derived from acrylonitrile and from 0% to 50% of units derived from an acrylic or styrene monomer, wherein the sum of the percentages (by weight) is equal to 100.

39. The composition according to claim 38, wherein the acrylic monomer is chosen from methyl (meth)acrylate and ethyl (meth)acrylate.

40. The composition according to claim 38, wherein the styrene monomer is chosen from α-methylstyrene and styrene.

41. The composition according to claim 6, wherein the at least one acrylic polymer powder comprises hollow particles of an expanded copolymer of vinylidene chloride and of acrylonitrile or of vinylidene chloride, of acrylonitrile and of methacrylate.

42. The composition according to claim 6, wherein the at least one acrylic polymer powder comprises particles having an internal cavity comprising at least one gas chosen from air, nitrogen, isobutane and isopentane.

43. The composition according to claim 6, wherein the at least one acrylic polymer powder comprises particles having a particle size ranging from 1 μm to 80 μm.

44. The composition according to claim 43, wherein the at least one acrylic polymer powder comprises particles having a particle size ranging from 10 μm to 30 μm.

45. The composition according to claim 6, wherein the at least one acrylic polymer powder other than the polymethyl methacrylate powder is present in an amount ranging from 0.05% to 2% by weight, relative to the total weight of the composition.

46. The composition according to claim 45, wherein the at least one acrylic polymer powder other than the polymethyl methacrylate powder is present in an amount ranging from preferably ranging from 0.1% to 1.2% by weight, relative to the total weight of the composition.

47. The composition according to claim 1, further comprising at least one polyurethane powder.

48. The composition according to claim 47, wherein the at least one polyurethane powder is a powder of a copolymer of hexamethylene diisocyanate and trimethylol hexyl lactone.

49. The composition according to claim 47, wherein the at least one polyurethane powder is present in an amount ranging from 0.5% to 30% by weight, relative to the total weight of the composition.

50. The composition according to claim 49, wherein the at least one polyurethane powder is present in an amount ranging from 5% to 15% by weight, relative to the total weight of the composition.

51. The composition according to claim 2, further comprising at least one N-acylated amino acid powder.

52. The composition according to claim 1, wherein the at least one N-acylated amino acid powder comprises at least one acyl group comprising from 8 to 22 carbon atoms.

53. The composition according to claim 52, wherein the at least one N-acylated amino acid powder comprises at least one acyl group chosen from 2-ethylhexanoyl, caproyl, lauroyl, myristoyl, palmitoyl, stearoyl, and cocoyl groups.

54. The composition according to claim 53, wherein the amino acid portion of the at least one N-acylated amino acid is chosen rom lysine, glutamic acid, and alanine.

55. The composition according to claim 53, wherein the at least one N-acylated amino acid powder is lauroyllysine.

56. The composition according to claim 1, wherein the at least one N-acylated amino acid powder is present in an amount ranging from 5% to 20% by weight, relative to the total weight of the composition.

57. The composition according to claim 56, wherein the at least one N-acylated amino acid powder is present in an amount ranging from 8% to 15% by weight, relative to the total weight of the composition.

58. The composition according to claim 1, further comprising barium sulfate.

59. The composition according to claim 57, wherein the barium sulfate is coated with at least one N-acylated amino acid powder.

60. The composition according to claim 59, wherein the at least one at least one N-acylated amino acid powder comprises at least one acyl group comprising from 8 to 22 carbon atoms

61. The composition according to claim 59, wherein the barium sulfate is coated with lauroyllysine.

62. The composition according to claim 58, wherein the barium sulfate is present in an amount ranging from 1% to 10% by weight, relative to the total weight of the composition.

63. The composition according to claim 63, wherein the barium sulphate is present in an amount ranging from 3% to 7% by weight, relative to the total weight of the composition.

64. The composition according to claim 1, further comprising at least one pulverulent dyestuff.

65. The composition according to claim 64, wherein the at least one pulverulent dyestuff is chosen from pigments and nacres.

66. The composition according to claim 65, wherein the at least one pulverulent dyestuff is present in an amount ranging from 0.5% to 40% by weight, relative to the total weight of the composition.

67. The composition according to claim 66, wherein the at least one pulverulent dyestuff is present in an amount ranging from 3% to 25% by weight, relative to the total weight of the composition.

68. The composition according to claim 1, further comprising at least one additional filler.

69. The composition according to claim 68, wherein the at least one additional filler is chosen from talc, mica, silica, kaolin, polyamide powders, poly-β-alanine powders, polyethylene powders, tetrafluoroethylene polymer powders, starch, boron nitride, silicone resin powders, hydroxyapatite, sericite, glass beads, and ceramic beads.

70. The composition according to claim 69, wherein the at least one additional filler is present in an amount ranging from 0.5% to 75% by weight, relative to the total weight of the composition.

71. The composition according to claim 70, wherein the at least one additional filler is present in an amount ranging from 5% to 60% by weight, relative to the total weight of the composition.

72. The composition according to claim 1, wherein the composition is free of compounds chosen from calcium carbonate, magnesium carbonate, magnesium hydrocarbonate, chalks, titanium dioxide, and metal soaps derived from organic carboxylic acids comprising from 8 to 22 carbon atoms.

73. The composition according to claim 1, wherein the composition comprises a total content of pulverulent compounds ranging from 80% to 99% by weight, relative to the total weight of the composition.

74. The composition according to claim 73, wherein the composition comprises a total content of pulverulent compounds ranging from 85% to 99% by weight, relative to the total weight of the composition.

75. The composition according to claim 1, further comprising at least one oil.

76. The composition according to claim 75, wherein the at least one oil is present in an amount ranging from 1% to 20% by weight, relative to the total weight of the composition.

77. The composition according to claim 76, wherein the at least one oil is present in an amount ranging from 2% to 15% by weight, relative to the total weight of the composition.

78. The composition according to claim 1, further comprising at least one cosmetic ingredient chosen from antioxidants, fragrances, preserving agents, neutralizers, surfactants, waxes, water, sunscreens, vitamins, moisturizers, self-tanning compounds, antiwrinkle active agents and deodorant active agents.

79. The composition according to claim 1, wherein the composition is anhydrous.

80. The composition according to claim 1, wherein the composition is in the form of a loose powder or a compact powder.

81. The composition according to claim 1, wherein the composition is in the form of a makeup powder or a skincare powder.

82. The composition according to claim 1, wherein the composition is in the form of a foundation, an eyeshadow, a makeup rouge, a concealer product, a body makeup product, a face care product, a body care product, and/or a deodorant powder.

83. A cosmetic makeup or therapeutic treatment process for the skin, comprising applying to the skin at least one cosmetic composition comprising,
at least one elastomeric organopolysiloxane powder;
at least one spherical filler; and
at least one N-acylated amino acid powder,
wherein the composition is in powder form.

84. A method for obtaining a deposit on skin comprising applying to the skin at least one cosmetic composition in the form of a powder comprising,
at least one elastomeric organopolysiloxane powder;
at least one spherical filler; and
at least one N-acylated amino acid powder,
wherein the deposit is transparent and/or masks imperfections in the skin relief and/or is natural and/or allows the grain of the skin to show through.
